(19) United States
(12) Patent Application Publication
(10) Pub. No.: US 2016/0361367 A1
(43) Pub. Date: Dec. 15, 2016

(54) PHARMACOPUNCTURE COMPOSITION FOR EYE HEALTH AND METHOD FOR MANUFACTURING THE SAME

(71) Applicant: Mi Kyung HA, Seongnam-si (KR)

(72) Inventor: Mi Kyung HA, Seongnam-si (KR)

(21) Appl. No.: 15/178,907

(22) Filed: Jun. 10, 2016

(30) Foreign Application Priority Data
Jun. 11, 2015 (KR) 10-2015-0082554

(51) Int. Cl. A61K 35/586; A61K 36/638; A61K 36/88; A61K 36/234; A61K 36/815; A61K 36/804; A61K 36/8965; A61K 36/40; A61K 36/77

(52) U.S. Cl. CPC A61K 35/586; A61K 36/40; A61K 36/638; A61K 36/88; A61K 36/77; A61K 36/815; A61K 36/804; A61K 36/8965; A61K 36/234

(57) ABSTRACT

The present invention relates to a pharmacopuncture composition for eye health that includes medicinal materials beneficial to the improvement of eye health and a method for manufacturing the same that is conducted according to the characteristics of the medicinal materials. The pharmacopuncture composition includes a mixed extract obtained from Cornic fructus, Ligustri fructus, Acori rhizome, Euphoria longana, Lycii fructus, Rehmanniae radix, Asparagi tuber, and Cnidii rhizoma through a water bath and mixed with a Testudinis carapax concentrated liquid.

### BACKGROUND OF THE INVENTION

Field of the Invention

The present invention relates to a pharmacopuncture composition for eye health and a method for manufacturing the same, and more particularly, to a pharmacopuncture composition for eye health that includes medicinal materials beneficial to the improvement of eye health and a method for manufacturing the same that is conducted according to the characteristics of the medicinal materials.

Background of the Related Art

As people spend a lot of time in watching images provided through various kinds of video equipment, they have had their visual impairment since they were the children, and with the increase of elderly population, further, degenerative diseases in eyes like cataract have been also increased. Therefore, the care and treatment of eyes should be needed in all ages.

In conformity with such need in eye health, oriental medicinal materials for eye health have been proposed even by the field of oriental medicine, but up to now, the oriental medicinal materials beneficial to eye health on the basis of herbology are simply mixed and eaten just as herbal medicinal preparations.

On the other hand, pharmacopuncture therapy is carried out by extracting an oriental medicinal composition (which is referred to as pharmacopuncture composition) on the basis of acupuncture priciple and herbology in the field of oriental medicine and by injecting the pharmacopuncture composition to acupuncture points, so that both of acupuncture effects and oriental medicinal material effects are all obtained to provide excellent therapy effects.

### SUMMARY OF THE INVENTION

Accordingly, the present invention has been made in view of the above-mentioned problems occurring in the prior art, and it is an object of the present invention to provide a pharmacopuncture composition for eye health that includes medicinal materials beneficial to the improvement of eye health and a method for manufacturing the same that is conducted according to the characteristics of the medicinal materials.

To accomplish the above-mentioned object, according to a first aspect of the present invention, there is provided a pharmacopuncture composition for eye health including a mixed extract obtained from Cornic fructus, Ligustri fructus, Acori rhizome, Euphoria longana, Lycii fructus, Rehmanniae radix, Asparagi tuber, and Cnidii rhizoma through a water bath.

According to the present invention, desirably, the mixed extract is added to a Testudinis carapax concentrated liquid.

According to the present invention, desirably, among Cornic fructus, Ligustri fructus, Acori rhizome, Euphoria longana, Lycii fructus, Rehmanniae radix, Asparagi tuber, and Cnidii rhizoma, if each of Ligustri fructus, Lycii fructus, and Rehmanniae radix has 10 parts by weight, Asparagi tuber, Cnidii rhizoma, Euphoria longana, Cornic fructus, and Acori rhizome have one of 8, 6, 4 and 2 parts by weight.

To accomplish the above-mentioned object, according to a second aspect of the present invention, there is provided a method for manufacturing a pharmacopuncture composition for eye health, the method including the steps of: washing Cornic fructus, Ligustri fructus, Acori rhizome, Euphoria longana, Lycii fructus, Rehmanniae radix, Asparagi tuber, and Cnidii rhizoma, respectively and conducting first pulverization for the medicinal materials; conducting second pulverization for the medicinal materials to 70 to 80 meshes through a hammer mill so that the medicinal materials have the same parts by weight as each other, and otherwise, if each of Ligustri fructus, Lycii fructus, and Rehmanniae radix has 10 parts by weight, Asparagi tuber, Cnidii rhizoma, Euphoria longana, Cornic fructus, and Acori rhizome have one of 8, 6, 4 and 2 parts by weight; putting the medicinal materials and purified water in a medicine boiling pot and conducting water bathing; and allowing the medicinal materials gasified to be liquefied by means of cooling to obtain a mixed extract from the medicinal materials.

According to the present invention, desirably, the time for water bath of the medicinal materials in the medicine boiling pot is 7 to 8 hours, and a water bath temperature is in the range of 70 to 80° C.

According to the present invention, desirably, the method further includes the steps of: stir-frying Testudinis carapax in such a manner as to sequentially raise a temperature in the range of 180 to 250° C. for about one hour; allowing the fried Testudinis carapax to be soaked in vinegar for two hours and washed; and putting and steaming the washed Testudinis carapax in a steamer for about 48 hours to extract a Testudinis carapax concentrated liquid with 17 to 20 degrees brix.

According to the present invention, desirably, the mixed extract and the Testudinis carapax concentrated liquid with 17 to 20 degrees brix are mixed in a volume ratio of 60:1 to 50:1.

### BRIEF DESCRIPTION OF THE DRAWINGS

FIG. 1 is a graph and table showing clinical trial example 1 of the present invention;

FIG. 2 is a graph and table showing clinical trial example 2 of the present invention;

FIG. 3 is a graph and table showing clinical trial example 3 of the present invention; FIG. 4 is a graph and table showing clinical trial example 4 of the present invention;

FIG. 5 is a graph and table showing clinical trial example 5 of the present invention;

FIG. 6 is a graph and table showing clinical trial example 6 of the present invention;

FIG. 7 is a graph and table showing clinical trial example 7 of the present invention;

FIG. 8 is a table showing clinical trial example 8 of the present invention; FIG. 9 is a graph and table showing clinical trial example 9 of the present invention;

FIG. 10 is a graph and table showing clinical trial example 10 of the present invention; and

FIG. 11 is a graph and table showing clinical trial example 11 of the present invention.

### DETAILED DESCRIPTION OF THE PREFERRED EMBODIMENTS

Hereinafter, an explanation on a pharmacopuncture composition for eye health and a method for manufacturing the same according to the present invention will be in detail given.

A pharmacopuncture composition for eye health according to the present invention includes total nine kinds of medicinal materials such as Cornic fructus, Ligustri fructus, Acori rhizome, Euphoria longana, Testudinis carapax, Lycii fructus, Rehmanniae radix, Asparagi tuber, and Cnidii rhizoma.

Referring to detailed effects of the medicinal materials, first, Cornic fructus is a dried fruit of Cornus officinalis Siebold et Zuccarini of the family Cornaceae, from which a seed is removed, and it is effective in improvements of liver and kidney functions. Particularly, it contains a large amount of vitamin A so that it is good for eye health. However, it contains toxic protein in its seed, thus causing kidney disorder.

Ligustri fructus is a fruit of Wax Leaf Privet of the family Oleaceae, and according to the illustrated book of Korean Medicinal Herbs, it is effective in visual impairment like weak sight or various eye diseases like glaucoma.

Acori rhizoma is a plant which is contained in the genus Arisaema erubescens Schott of the family Acorus gramineus Solander, and in this case, the rhizome is used as a medicinal material. Acori rhizoma is effective in treatment of red eyes or corneal ulcer. However, it has ‘asarone’ as a main ingredient thereof, which is toxic, so that if it is excessively eaten in the form of a concentrated extract or raw root, it causes side effects like vomiting or cancer-causing.

Euphoria longana is a dried fruit of Dimocarpus longan Lour of the family Sapindus mukorossi Gaertntner and has abundant vitamins including vitamin A and minerals, so that if it is eaten for a long period of time, it is good for eye and ear health. According to Sasang constitution medicine, it has warm properties, and when it is much eaten, it is not good for So-yang or Tae-yang person.

Testudinis carapax is a plastron or carapace of a turtle (Freshwater tortoise) and is very effective in strengthening energy of liver, kidney and heart.

Lycii fructus is a fruit of Lycium chinense Miller of the family Solanaceae and is generally used as nutrients and tonics. Especially, it has effects for preventing the disorder in liver functions, for recovering damaged liver, for accelerating the regeneration of liver cells, and for suppressing fatty liver, so that it is directly and indirectly related to the functional or organic health of liver.

Rehmanniae radix is the steamed root of Rehmannia glutinosa Liboschitz ex Steudel of the family Scrophulariaceae, and according to Modern practice Chinese herbal medicine (published by People's medical publishing house in China), it has immunity enhancing effects, hemostatic effects, blood glucose dropping effects, and protection effects from chromosomal damage of bone marrow cells. Further, it has effects of nutrients and tonics and liver function protection effects. However, it has plenty of nutrients and oil, and accordingly, if it is eaten for a long period of time, it causes digestive problems.

Asparagi tuber is a tuberous root of a perennial plant of the family Liliaceae, from which a cork layer is removed, and it is effective in diuresis, tonics, antitussives, and thirst quenching. According to Modern practice Chinese herbal medicine, it has anti-bacterial effects, anti-aging effects, and insect killing effects. However, it has very cold properties, thus giving side effects to a person having a cold body or bowl trouble.

Cnidii rhizome is a root stock of a perennial plant of the family Apiaceae (Umbelliferae) and is effective in accelerating blood circulation through strengthening of blood vessel motions. However, if it is eaten by a person having a lot of fever, it causes side effects like a headache.

As mentioned above, Acori rhizome, Euphoria longana, Ligustri fructus, and Cornic fructus among the nine kinds of medicinal materials contain pharmaceutical ingredients related directly in eye health, and Testudinis carapax, Lycii fructus, Rehmanniae radix, and Asparagi tuber strength the functions of liver and kidney organs having influences on eye health, which means they contain pharmaceutical ingredients capable of improving eye health.

Further, Cnidii rhizome accelerates blood circulation to indirectly support the movements of the pharmaceutical ingredients and the expression of pharmaceutical ingredients effects.

The pharmacopuncture composition for eye health according to the present invention includes Acori rhizome, Euphoria longana, Ligustri fructus, and Cornic fructus as base medicinal materials and selectively adds Testudinis carapax, Lycii fructus, Rehmanniae radix, Asparagi tuber and Cnidii rhizome according to the health state of a patient.

Particularly, if the patient have excessively cold or hot body constitutions so as to prevent the generation of side effects, Asparagi tuber, Cnidii rhizome and Euphoria longana dividedly contain parts by weight step by step, and on the other hand, Cornic fructus and Acori rhizome having toxic ingredients quantitatively contain parts by weight step by step.

In more detail, the eight kinds of medicinal materials except Testudinis carapax contain the same parts by weight as each other and obtain their extract through a distillation and extraction method as will be discussed later. If the eight kinds of medicinal materials except Testudinis carapax have 100 g by weight, respectively, Asparagi tuber, Cnidii rhizome, Euphoria longana, Cornic fructus and Acori rhizome reduce parts by weight to 80 g, 60 g, and 40 g step by step, and after that, they are injected into a medicine boiling pot for distillation and extraction.

Hereinafter, an explanation on a method for manufacturing a pharmacopuncture composition for eye health according to the present invention will be in more detail given through the division of pre-processing and mixing and post-processing.

The pre-processing is a process for obtaining an extract from medicinal materials, and the eight kinds of medicinal materials except Testudinis carapax is subjected to the distillation and extraction treatment typically used when the pharmacopuncture composition is extracted from mixed medicinal materials.

After the medicinal materials are first washed, they are pulverized, which makes the extraction ease, and since precision and quantitativeness are required in the present invention, the medicinal materials are secondarily pulverized to 70 to 80 meshes through a hammer mill after the first pulverization. Next, a quantitative process is carried out.

After that, the pulverized medicinal materials are quantitatively injected into the lower portion of a reaction tank of the medicine boiling pot, and after they are soaked in purified water in consideration of their volumes, they are subjected to water bathing. In this case, the purified water is obtained by means of sequential filtering through a membrane filter, a carbon filter, and a ceramic filter.

The plant medicinal materials generally contain volatile ingredients, and accordingly, they are subjected to the water bathing through a steam heating type medicine boiling pot, which is capable of minimizing the change in pharmaceutical ingredients caused by the volatilization and capable of filtering the oil and fat contained in the medicinal materials.

The time for water bath of the medicinal materials is 7 to 8 hours, which is longer than general bath time, and a water bath temperature is kept to a temperature of 70 to 80° C.

Testudinis carapax is the only animal medicinal material among the medicinal materials used in the present invention, which is subjected to a separate extraction process. First, Testudinis carapax is stir-fried in such a manner as to sequentially raise a temperature in the range of 180 to 250° C. for about one hour, soaked in vinegar for two hours and washed, is put and steamed in a steamer for about 48 hours, and is extracted to a form of a Testudinis carapax concentrated liquid with 17 to 20 degrees brix.

The mixing and post-processing is carried out by adding the Testudinis carapax concentrated liquid to the extract (which is referred to as mixed extract) obtained through the distillation and extraction method. Next, the mixture is sterilized and heated, and after the sterilized mixture is put into sealed containers for injections, it is kept at a refrigerator.

In this case, the mixing ratio of the mixed extract to the Testudinis carapax concentrated liquid has a volume ratio of 60:1 to 50:1. That is, 100 cc of the Testudinis carapax concentrated liquid is added to 6000 to 5000 cc of the mixed extract.

The pharmacopuncture therapy through the pharmacopuncture composition manufactured by the above-mentioned method has Cheon-ju and Ye-pung as base acupuncture points and additionally has acupuncture points which strengthen eyes, optic nerves, liver, and kidney functions selected from the acupuncture points located on the arteries around the spine so as to insert magnetic needles and inject the medicinal materials into them.

Further, an extract like hominis placenta or Cervus elaphus sibericus, which is existing pharmacopuncture composition injected into general acupuncture points, is injected into the acupuncture points around eyes, as an auxiliary therapy in the pharmacopuncture therapy according to the present invention.

### <Clinical Trial Examples>

The pharmacopuncture therapy with the pharmacopuncture composition according to the present invention was carried out for patients who have visual impairment two to three times every week for 4 to 8 weeks on base acupuncture points, Cheon-ju and Ye-pung, and next, the changes of their visual acuity were measured.

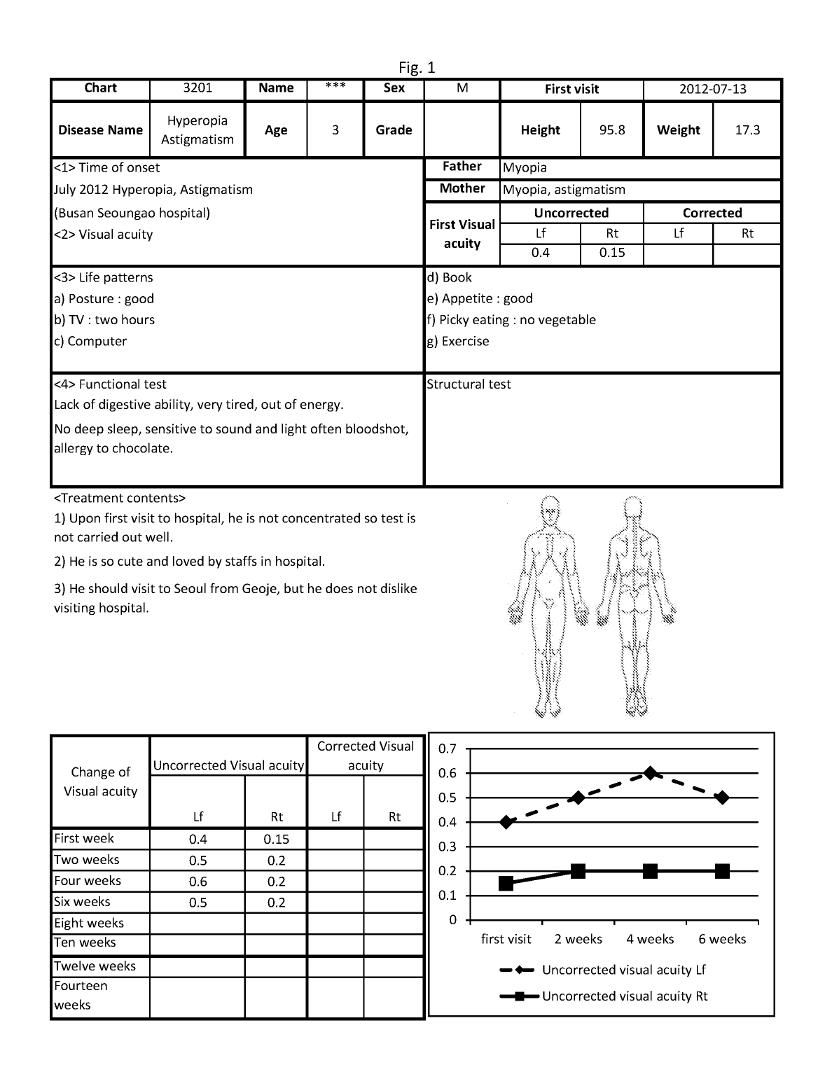

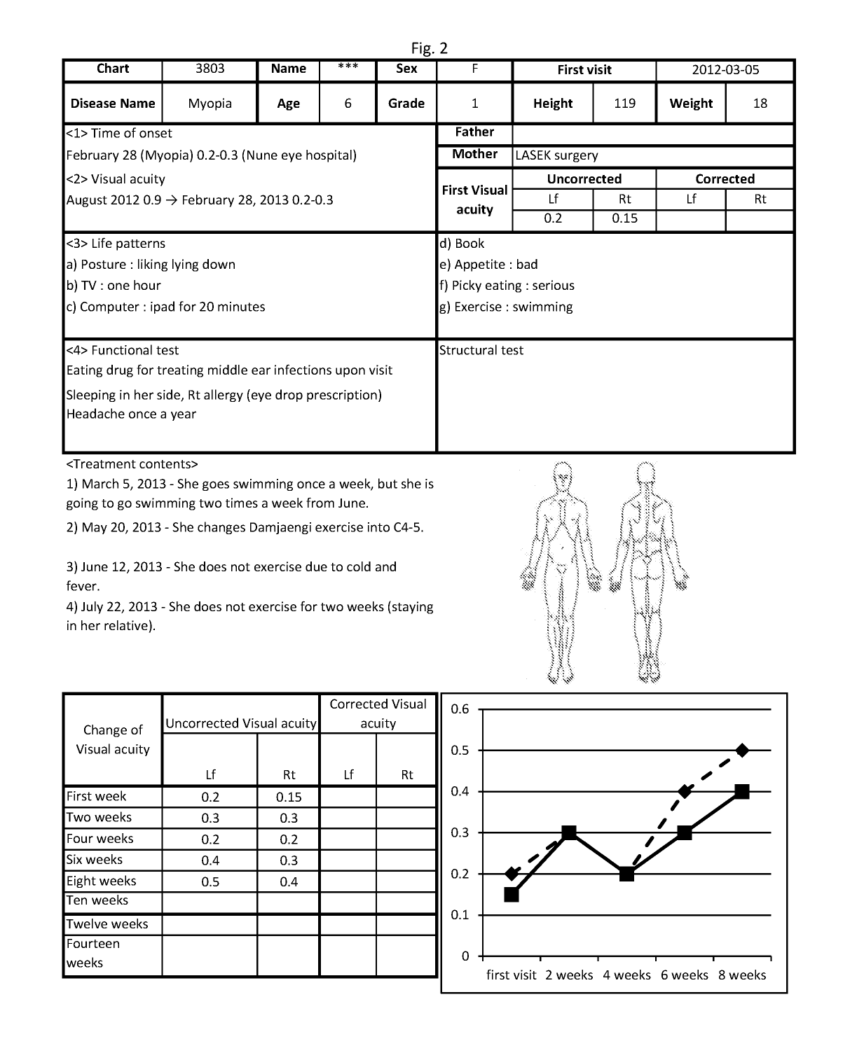

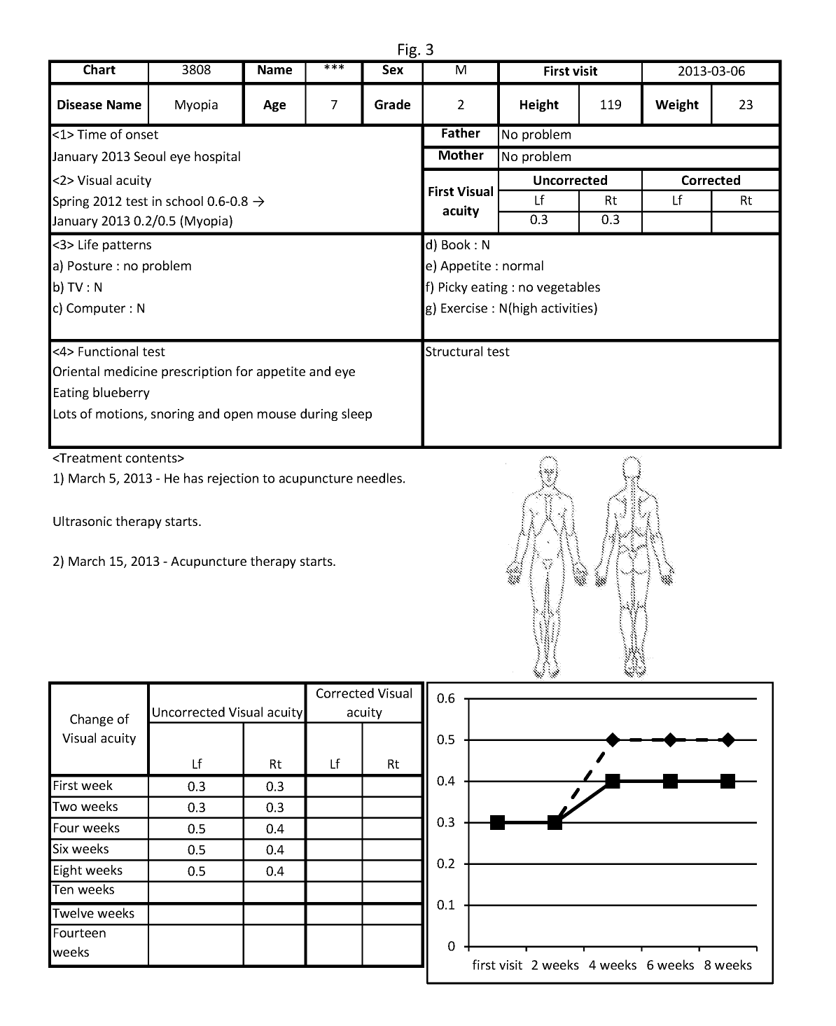

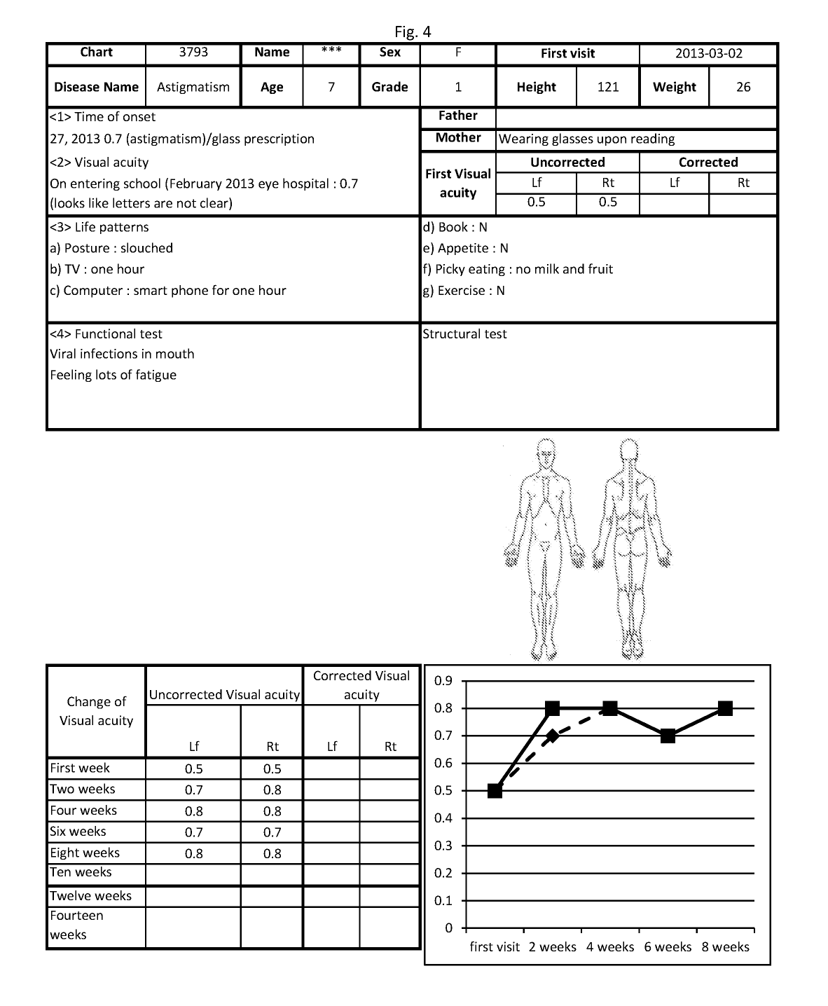

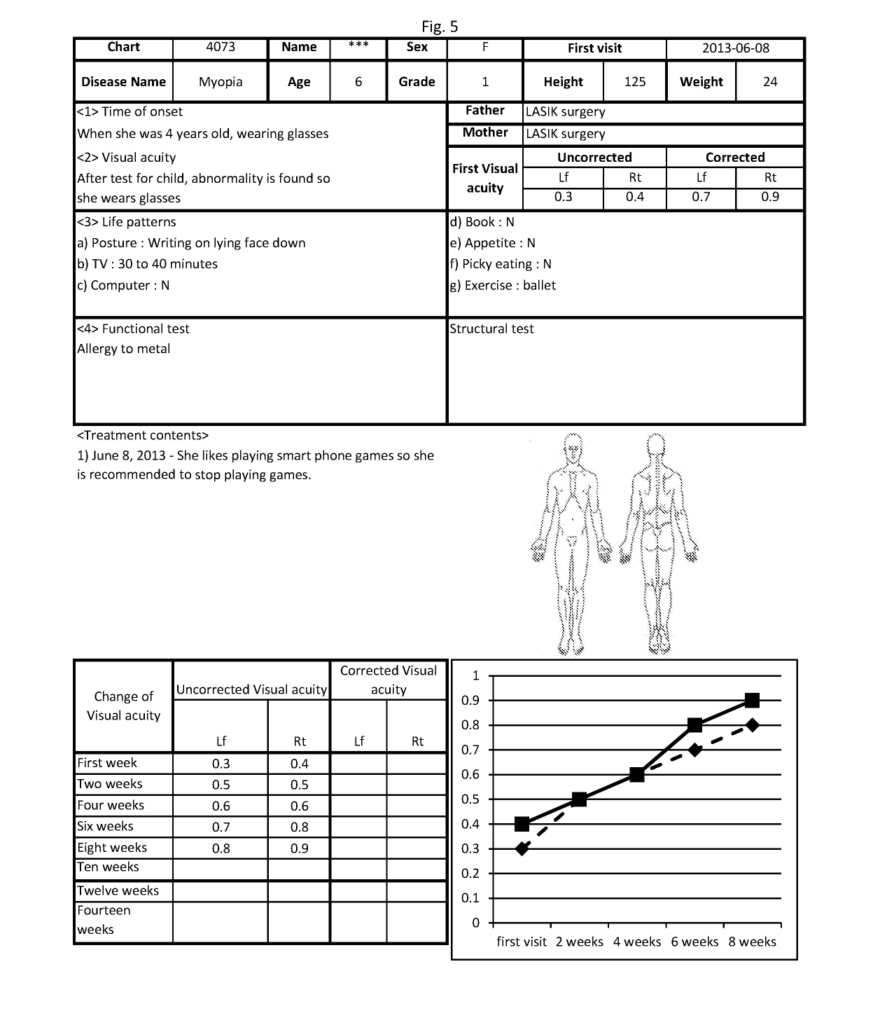

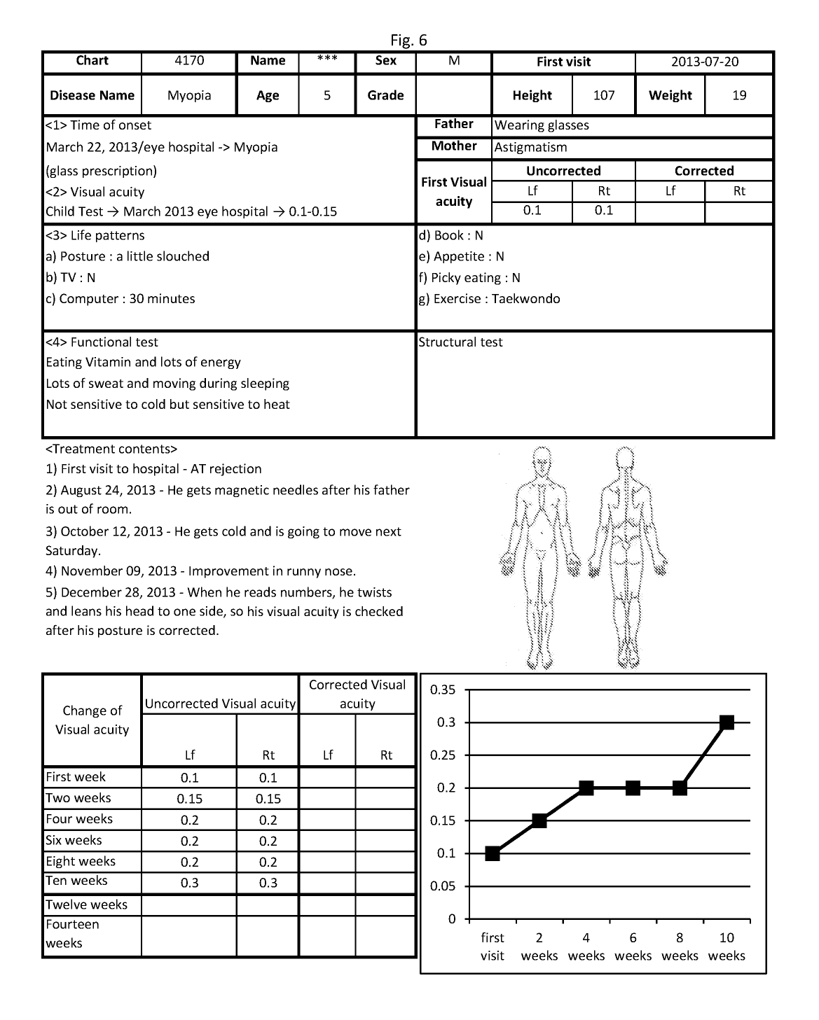

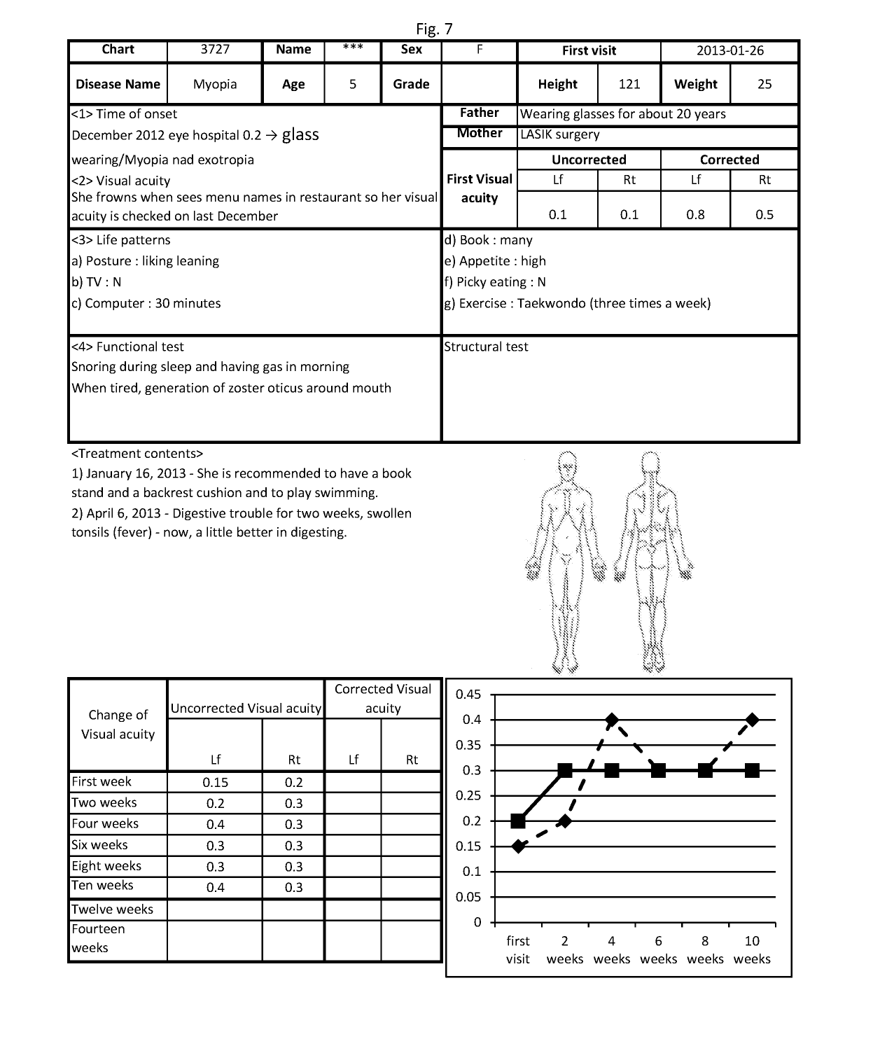

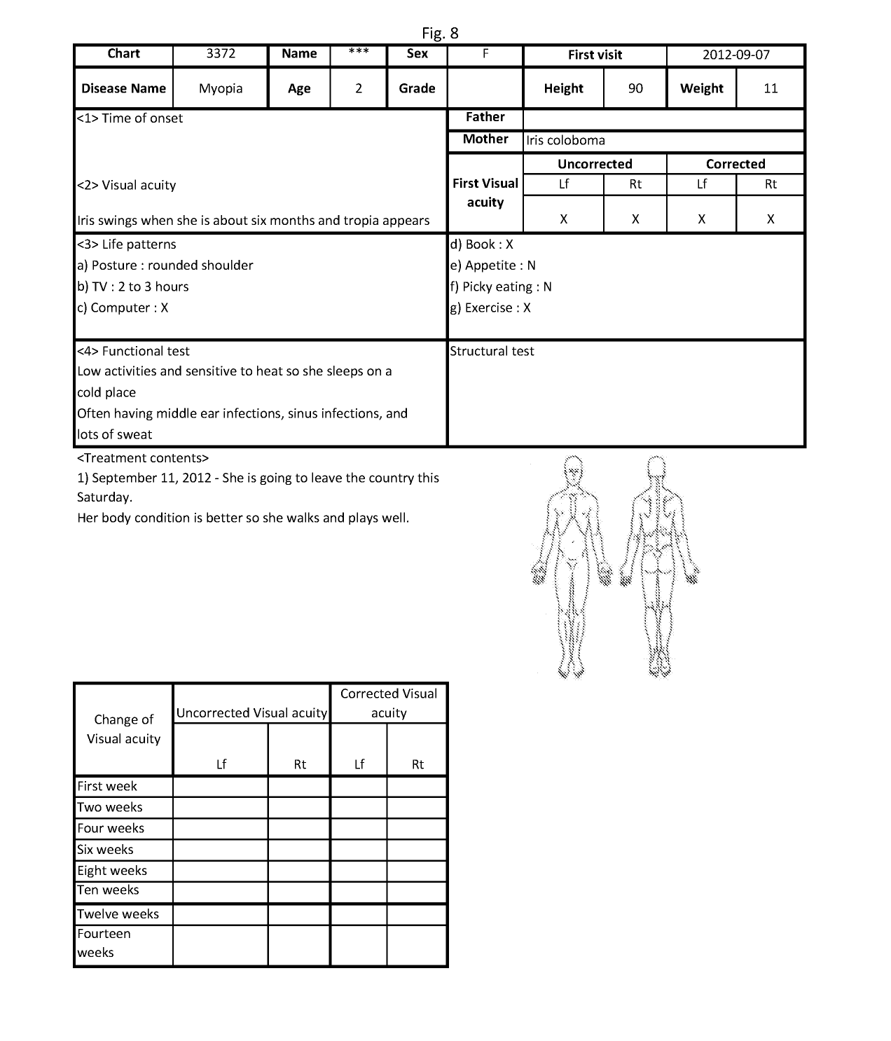

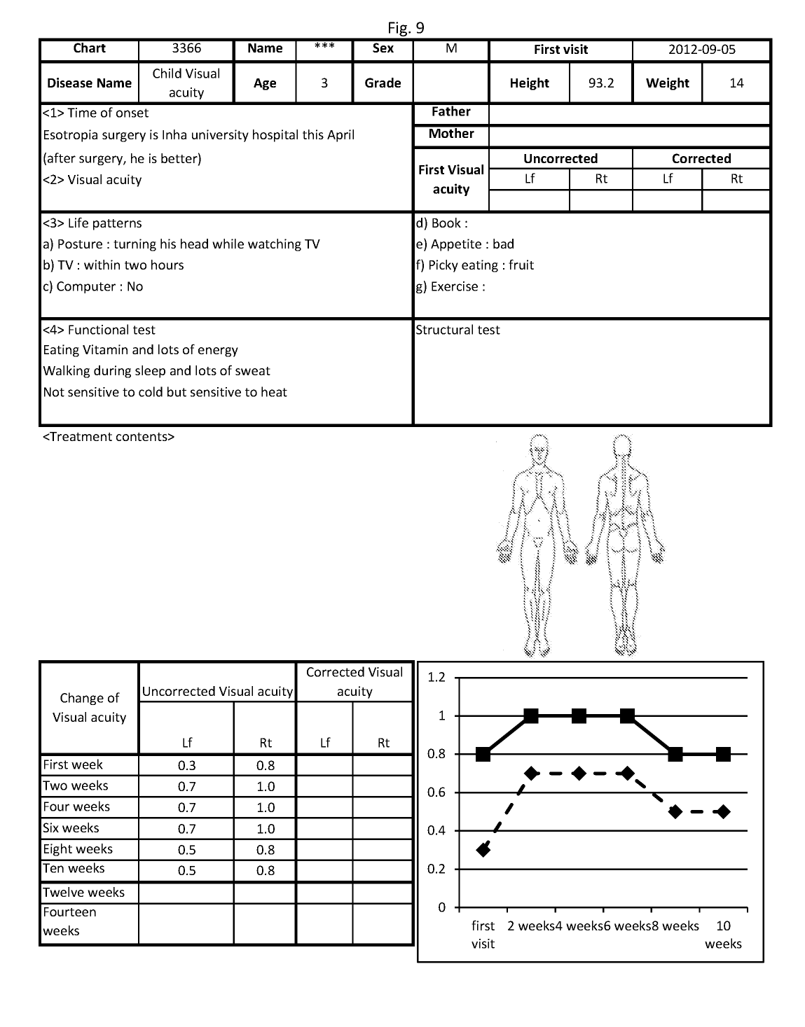

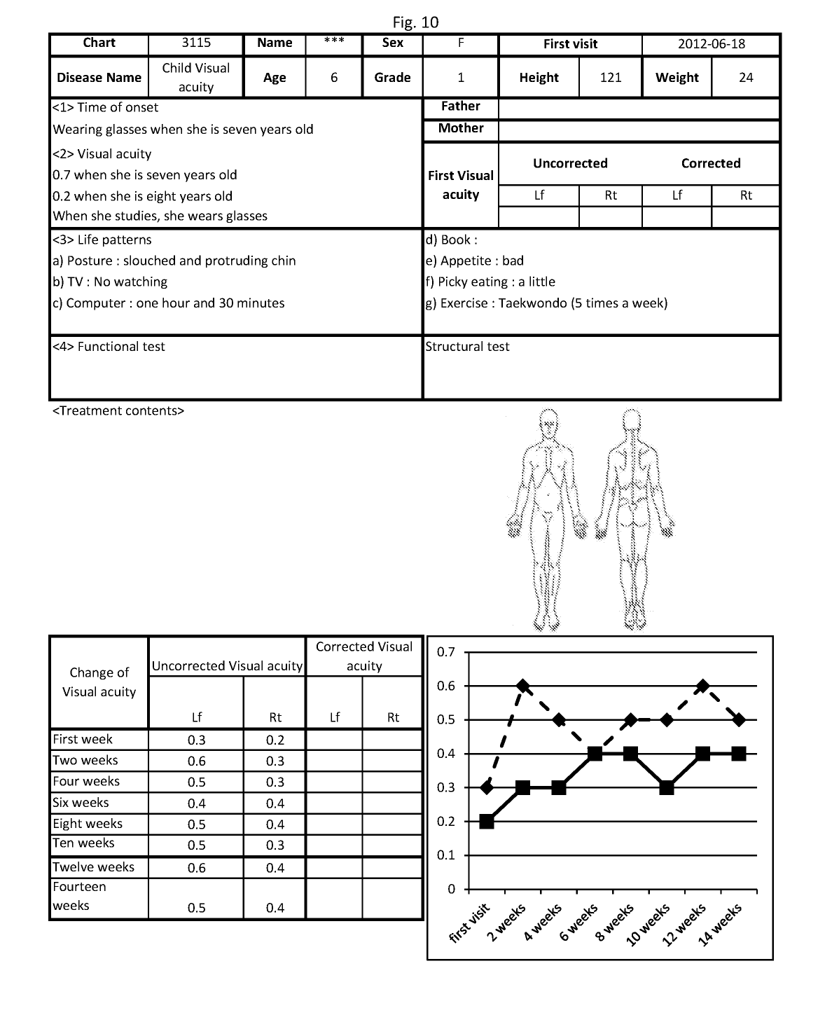

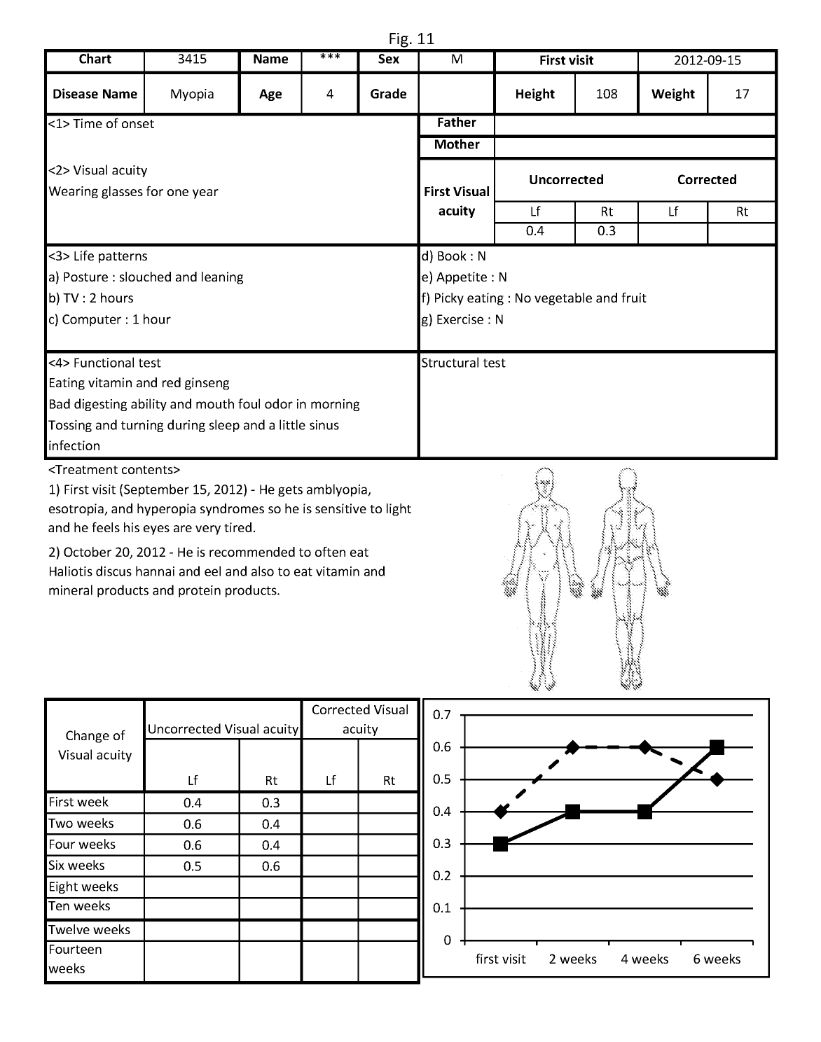

As shown in FIG. 1 to FIG. 11, the pharmacopuncture composition for eye health and the method for manufacturing the same according to the present invention includes Acori rhizome, Euphoria longana, Ligustri fructus, and Cornic fructus as base medicinal materials and selectively adds Testudinis carapax, Lycii fructus, Rehmanniae radix, Asparagi tuber and Cnidii rhizome, so that acupuncture points effective in improving eye health are stimulated, without having any digesting process, thereby achieving excellent eye health improvements and rapid treatment effects.

While the present invention has been described with reference to the particular illustrative embodiments, it is not to be restricted by the embodiments but only by the appended claims. It is to be appreciated that those skilled in the art can change or modify the embodiments without departing from the scope and spirit of the present invention.

What is claimed is:

1. A pharmacopuncture composition for eye health, comprising a mixed extract obtained from Cornic fructus, Ligustri fructus, Acori rhizome, Euphoria longana, Lycii fructus, Rehmanniae radix, Asparagi tuber, and Cnidii rhizoma through a water bath.

2. The pharmacopuncture composition for eye health according to claim 1, wherein the mixed extract has a Testudinis carapax concentrated liquid with 17 to 20 degrees brix added thereto in a volume ratio of 60:1 to 50:1.

3. The pharmacopuncture composition for eye health according to claim 1, wherein among Cornic fructus, Ligustri fructus, Acori rhizome, Euphoria longana, Lycii fructus, Rehmanniae radix, Asparagi tuber, and Cnidii rhizoma, if each of Ligustri fructus, Lycii fructus, and Rehmanniae radix has 10 parts by weight, Asparagi tuber, Cnidii rhizoma, Euphoria longana, Cornic fructus, and Acori rhizome have one of 8, 6, 4 and 2 parts by weight.

4. A method for manufacturing a pharmacopuncture composition for eye health, the method comprising the steps of: washing Cornic fructus, Ligustri fructus, Acori rhizome, Euphoria longana, Lycii fructus, Rehmanniae radix, Asparagi tuber, and Cnidii rhizoma, respectively and conducting first pulverization for the medicinal materials; conducting second pulverization for the medicinal materials to 70 to 80 meshes through a hammer mill so that the medicinal materials have the same parts by weight as each other, and otherwise, if each of Ligustri fructus, Lycii fructus, and Rehmanniae radix has 10 parts by weight, Asparagi tuber, Cnidii rhizoma, Euphoria longana, Cornic fructus, and Acori rhizome have one of 8, 6, 4 and 2 parts by weight; putting the medicinal materials and purified water in a medicine boiling pot and conducting water bathing; and allowing the medicinal materials gasified to be liquefied by means of cooling to obtain a mixed extract from the medicinal materials.

5. The method according to claim 4, wherein the time for water bath of the medicinal materials in the medicine boiling pot is 7 to 8 hours, and a water bath temperature is in the range of 70 to 80° C.

6. The method according to claim 4, further comprising the steps of: stir-frying Testudinis carapax in such a manner as to sequentially raise a temperature in the range of 180 to 250° C. for about one hour; allowing the fried Testudinis carapax to be soaked in vinegar for two hours and washed; and putting and steaming the washed Testudinis carapax in a steamer for about 48 hours to extract a Testudinis carapax concentrated liquid with 17 to 20 degrees brix.

7. The method according to claim 6, wherein the mixed extract and the Testudinis carapax concentrated liquid with 17 to 20 degrees brix are mixed in a volume ratio of 60:1 to 50:1.
